(19) United States
(12) Patent Application Publication
(10) Pub. No.: US 2016/0228304 A1
(43) Pub. Date: Aug. 11, 2016

(54) Comfort Nights Overnight Pad

(71) Applicant: Coreena Orechva, Revere, MA (US)

(72) Inventor: Coreena Orechva, Revere, MA (US)

(21) Appl. No.: 14/954,969

(22) Filed: Nov. 30, 2015

Related U.S. Application Data
(60) Provisional application No. 62/085,456, filed on Nov. 28, 2014.

(51) Int. Cl. A61F 13/475; A61F 13/472

(52) U.S. Cl. CPC A61F 13/4756; A61F 13/472; A61F 13/475; A61F 2013/16

(57) ABSTRACT

A contoured and elongated sanitary napkin or pad that conforms to the natural curves of the female body, primarily in the groin and buttock area, and is configured expressly for nighttime use and extremely heavy flow days wherein the pad comprises a series of raised, and shaped channels positioned throughout the surface of the pad to contain leaks, even when the wearer is laying on her side.

### CLAIM OF PRIORITY

This patent application claims priority under 35 USC 119 (e) (1) from U.S. Provisional Patent Application Ser. No. 62/085,456 filed Nov. 28,2014, of common inventorship herewith entitled, “Comfort Nights,” which is incorporated herein by reference as though the same were set forth in its entirety.

### FIELD OF THE INVENTION

The present invention pertains to the field of feminine hygiene, and more specifically to the field of sanitary napkins.

### BACKGROUND OF THE INVENTION

The prior art has put forth several designs for sanitary napkins. Among these are:

U.S. Pat. No. 7,597,690 to Toshiyuki Tanio, Wataru Yoshimasa, Kenichiro Kuroda and Shinobu Fujikawa describes a sanitary napkin including a surface element that is formed of a liquid permeable top sheet covering a liquid absorbent layer. The surface element is raised from the skin side surface of a napkin body by an elastic force of an elastic member to form a protuberance having a front end and a rear end. The protuberance fits in the intergluteal cleft and prevents rearward leakage of menstrual blood.

U.S. Pat. No. 6,524,291 to Camilla Bjorkland, Urban Widlund, Ann Samuelsson, Solgun Devrik and Anders Gustafsson describes an absorbent article such as a sanitary napkin, an incontinence guard or a panty liner. The article has a substantially elongated shape with a longitudinal direction and a transverse direction and also exhibits two side edges, a front edge and a rear edge, a front portion and a rear portion, and a crotch portion situated between the front portion and the rear portion. The article exhibits a liquid impervious surface. At least within the front portion and the crotch portion, the article exhibits high bending stiffness against bending along transversely extending bending lines, whereby the front portion and the crotch portion exhibit high shape permanence during use of the article. The front portion of the article is angled in relation to the crotch portion in a direction towards the liquid pervious surface, whereby the front portion forms an angle towards the crotch portion which is substantially permanent during use.

U.S. Pat. No. 4,804,380 to Frederich O. Lassen, Anna Cesco-Cancian and Dawn M. Huffman describes a three dimensionally shaped, externally worn feminine protection device which has been mechanically shaped by folding, molding or other forming techniques in such a way that it has a raised portion located within the back one half to two thirds of the device that functions to cause the pad to readily fit to and generally align itself within the inverted V shaped regions of the woman's lower abdominal region, vulva, perineum and the anterior portion between the buttocks. The forward portion of the device of the invention generally is flattened in use to cover externally the area of the pubic mons and the area exterior of the clitoris. The back portion of the device is formed in a peak like shape which readily adjusts to and molds to the inverted V shape of the rear portion of the labia, the perineum and the forward portion of the area between the buttocks.

None of these prior art references describe the present invention.

### SUMMARY OF THE INVENTION

It is an object of the present invention to provide a contoured and elongated sanitary napkin or pad that conforms to the natural curves of the female body, primarily in the groin and buttock area, and is configured expressly for nighttime use and extremely heavy flow days. The sanitary napkin design of the present invention can also be used for daytime use, as well. The primary focus of the sanitary napkin of the present invention, and the focus of this patent application, is for protection of the groin and buttock area of the female anatomy.

### BRIEF DESCRIPTION OF THE DRAWINGS

FIG. 1 is a prototypical top down diagonal view of the present invention with raised channels and side wings that attach to the undergarment for security and help to provide maximum protection for a female wearer.

FIG. 2 is a prototypical top down diagonal view of the present invention with raised channels that attach to the undergarment for security and help to provide maximum protection for a female wearer. This embodiment does not have side wings that attach to the undergarment.

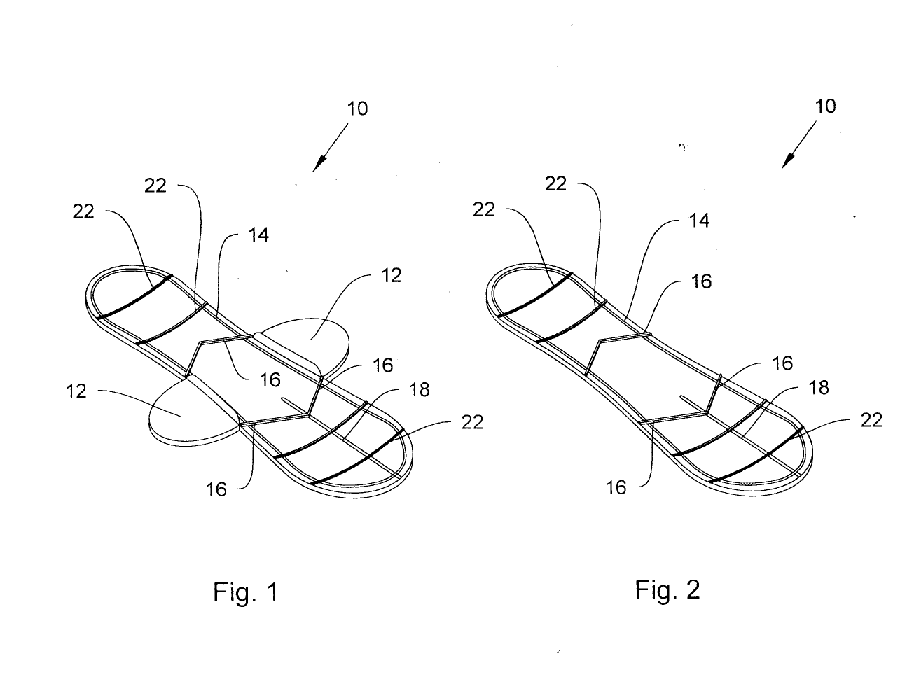

### DETAILED DESCRIPTION OF THE INVENTION

A regular menstrual cycle or period is a normal fact of life for most healthy adolescent girls and adult women. This monthly shedding of the lining of the uterus is an important aspect of a woman's fertility. Providing a cushiony nutrient rich bed for a fertilized egg to grow into a baby, the uterine lining slowly develops during the course of a month in anticipation of the egg's arrival. Although an egg is released every month from one of a woman's two ovaries, the majority of time this egg remains unfertilized. Simply dissolving after being dropped from the ovary, the egg and the lining are released from the body typically over the course of three to five days. A woman's menstrual cycle can begin as early as the age of nine and can last well into her fifties.

Most women control the flow from her periods by wearing either a sanitary napkin or a tampon. Sanitary napkins are configured to provide external protection and are applied directly to the wearer's undergarment while tampons are designed to be worn internally, absorbing moisture before it exits the body. Regardless which form of hygiene protection a woman prefers, both sanitary napkins and tampons provide female consumers a simple means of absorbing excess fluids, enabling them to remain clean, dry and comfortable during her monthly cycle.

Although tampons and sanitary napkins are necessary items for most women, there are those times when these products fail to provide adequate protection. While tampons can quickly become saturated with moisture, standard sanitary napkins often fail to provide enough coverage, particularly in the groin area, front and back of the pad. The flat rectangular shape of sanitary napkins often fails to conform to the natural curves of the female body, mainly in the groin and buttock area, causing leaks to occur. Many women experience heavy menstrual flow during her period, especially during the first few days. During the evening, when one is engaged in deep sleep for several uninterrupted hours, excessive menstrual flow can result in clothing and bedding being soiled with unsightly and hard to remove stains. This soiling is especially likely to happen if one sleeps on her back, stomach or tosses and turns during the night, as the flat design of sanitary napkins can result in excessive leakage. Waking in the morning to find one's clothing and bedding wet and stained is uncomfortable and frustrating.

Millions of females between the ages of approximately thirty five to forty nine years old suffer from a condition known as menorrhagia. Typically brought on by hormonal changes, menorrhagia is a condition of abnormally excessive menstrual bleeding. For women who experience heavy bleeding, protecting her undergarments, clothing and bedding from stains and wetness while maintaining a level of dryness and comfort is nearly impossible.

The present invention, hereinafter referred to as the Comfort Nights Overnight Pad, or simply Comfort Nights, is a contoured and elongated sanitary napkin or pad that conforms to the natural curves of a female body and is configured expressly for nighttime use and extremely heavy flow days. The Comfort Nights provide a female with reliable sanitary protection during her monthly period, particularly during hours of slumber. The Comfort Nights conforms to the shape of the frontal, middle and rear female anatomy. The present invention encompasses a significantly larger area than traditional sanitary pads.

The Comfort Nights is manufactured of super absorbent cotton/foam like material and backed in flexible, leak proof plastic. The uppermost material of this pad is constructed to pull moisture from the surface of the pad and lock it within the pad's absorbent layers. A sticky adhesive composite with or without the wings coats the underside of the Comfort Nights and adheres the pad to the wearer's undergarment. The Comfort Nights includes adhesively attachable wing components that wrap around the crotch panel of the wearer's underwear and further secure the present invention in place. A protective paper backing that covers this adhesive portion of the present invention is removed prior to use.

Please refer to the Figures. Generally hourglass in shape with a soft outer raised edge 14 around the entire perimeter of the pad, the Comfort Nights is tapered inwards toward the center of the pad to accommodate the crotch panel of the wearer's undergarment. The present invention measures approximately twelve inches in length and five inches in width at its widest point at each end of the Comfort Night. The center section of the present invention measures approximately three inches in width. Larger and smaller versions of the Comfort Nights are manufactured to accommodate females of different body types and statures.

Incorporated into the Comfort Nights is a series of raised channels that prevent liquid matter from seeping from the ends or sides of the pad and direct this liquid back on the pad where it is absorbed and held. The groin area sides 12 are raised higher than the outer border in order to “cup” the shape of the groin area. In the contoured center groin area 12 of the pad, raised and curved channels are positioned lengthwise on each side of the pad and configured to comfortably wrap around the exterior sides of the female genitalia thus creating a barrier that provides maximum leak protection when laying sideways. The curved front and back end of the pad comprise a semicircular raised channel that separates these sections from the center of the pad.

The semicircular front end of the pad measures slightly smaller in width than the rear circular portion of the pad. A set of arrow shaped channels 16 are centrally positioned in the front and back ends that function to contain leaks, directing moisture to the center of the pad. The rounded and wider rear section of the Comfort Nights encompasses the wearer's body between the base of her vagina and anus and rests flush over her buttocks. A slope like raised channel 18 starting low and thin at the groin area and gradually getting higher and wider toward the end of the napkin, so the napkin sits flush to the shape and curve of the buttocks. The wide slope like vertical channel 18 runs the length of the center of this portion of the pad, culminating in the circular outer channel 14 and two sets of two diagonal inwardly recessed channels 22 are spaced apart on each side of the pad. Two sets of arrow shaped channels 16 are positioned directly between the center portion of the pad and the back end the pad. For safe handling and disposal, each Comfort Nights is individually wrapped within sterile paper.

Application and use of the Comfort Nights is very simple and straight forward. A user removes the Comfort Nights from its sanitary outer wrapping, discarding the wrapping in any trash receptacle. The user unfolds the compacted pad and adheres the pad to the crotch panel of her underwear by means of the adhesive backing which coats the underside of the present invention. With the Comfort Nights affixed to her undergarment, the user pulls up her underwear, allowing the sanitary napkin to naturally conform to the shape of her body. As such, the front end of the Comfort Night extends up and over the groin area, while the center portion of the pad gently cups the wearer's crotch, hugging the body and encompassing the vaginal area completely. The groin area sides 12 are raised higher than the outer border 14 in order to “cup” the shape of the groin area. The curved back end of the pad rests over the shape of the buttocks, conforming to the wearer's natural curves. With the Comfort Nights properly applied and in place, the user then retires for the evening. The Comfort Nights also is worn during days of particularly heavy menstrual flow. Easily removed and replaced, The Comfort Nights is changed as often as necessary to maintain a feeling of comfort and freshness.

The Comfort Nights' form fitting surface along with integrated raised channels prevent wetness from seeping whether the wearer is laying supine on her back, sleeping on her stomach or tossing and turning throughout the evening. Gently extending from a wearer's groin to her buttocks, the ultra absorbent Comfort Nights encompass the area of the body where leaks most often occur. The Comfort Nights also protect the user's clothing and bedding from the unsightly and embarrassing stains which often occur when moisture leaks from the ends or sides of the pad. Compact in size, the present invention easily fits into any purse, briefcase, backpack, blouse or pant pocket.

Although this invention has been described with respect to specific embodiments, it is not intended to be limited thereto and various modifications which will become apparent to the person of ordinary skill in the art are intended to fall within the spirit and scope of the invention as described herein taken in conjunction with the accompanying drawings and the appended claims.

1. A contoured and elongated sanitary napkin or pad that conforms and fits to the natural curves of the female body and is configured expressly for nighttime use and extremely heavy flow days, comprising: a multiple layered pad of super absorbent cotton or foam like material and backed in flexible, leak proof plastic, wherein the uppermost layer of material is constructed to pull moisture from the surface of the pad and lock it within the pad's absorbent layers, and wherein a sticky adhesive composite coats the underside of the pad and adheres the pad to the wearer's undergarment further comprising a raised outer edge and raised channels that prevent liquid from seeping from the ends or sides of the pad and direct this liquid back onto the pad where it is absorbed and held.

2. The pad of claim 1 further comprising adhesively attachable wing components that wrap around the crotch panel of the wearer's underwear and further secure the pad in place, wherein a protective paper backing covers the adhesive portion of the pad and is removed prior to use.

3. The pad of claim 1, wherein the pad is hourglass in shape with a soft outer raised edge around the entire perimeter of the pad, wherein the pad is tapered inwards toward the center of the pad to accommodate the crotch panel of the wearer's undergarment.

4. The pad of claim 1, wherein the pad measures approximately twelve inches in length and five inches in width at its widest point, and wherein the center section of the pad measures approximately three inches in width.

5. The pad of claim 1, available in larger and smaller versions to accommodate females of different body types and statures.

6. The pad of claim 1 further wherein the groin area sides are raised higher than the outer border in order to “cup” the shape of the groin area.

7. The pad of claim 6, further comprising raised and curved channels in the center groin area of the pad positioned lengthwise on each side of the pad and configured to comfortably wrap around the exterior sides of the female genitalia thus creating a barrier that provides maximum leak protection when laying sideways.

8. The pad of claim 7, further comprising a pair of semicircular raised channels near the curved front of the pad and near the curved back of the pad that separates the back of the pad and the front of the pad from the center of the pad.

9. The pad of claim 8, further comprising a plurality of arrow shaped channels centrally positioned in the front and back ends that function to contain leaks, directing moisture to the center of the pad.

10. The pad of claim 9, wherein the curved front end of the pad measures slightly smaller in width than the rear curved portion of the pad.

11. The pad of claim 10, further comprising a sloped, raised channel starting low and thin at the groin area and gradually getting higher and wider toward the rear end of the napkin, so the napkin sits flush to the shape and curve of the buttocks, wherein the raised channel runs the length of the center of this portion of the pad, culminating in the circular outer channel and two sets of arrow shaped channels.

12. The pad of claim 2 further wherein the groin area sides are raised higher than the outer border in order to “cup” the shape of the groin area.

13. The pad of claim 12, further comprising raised and curved channels in the center groin area of the pad positioned lengthwise on each side of the pad and configured to comfortably wrap around the exterior sides of the female genitalia thus creating a barrier that provides maximum leak protection when laying sideways.

14. The pad of claim 13, further comprising a pair of semicircular raised channels near the curved front of the pad and near the curved back of the pad that separates the back of the pad and the front of the pad from the center of the pad.

15. The pad of claim 14, further comprising a plurality of arrow shaped channels centrally positioned in the front and back ends that function to contain leaks, directing moisture to the center of the pad.

16. The pad of claim 15, wherein the curved front end of the pad measures slightly smaller in width than the rear curved portion of the pad.

17. The pad of claim 16, further comprising a sloped, raised channel starting low and thin at the groin area and gradually getting higher and wider toward the rear end of the napkin, so the napkin sits flush to the shape and curve of the buttocks, wherein the raised channel runs the length of the center of this portion of the pad, culminating in the circular outer channel and two sets of arrow shaped channels.
